(19) United States
(12) Patent Application Publication
(10) Pub. No.: US 2006/0100170 A1
(43) Pub. Date: May 11, 2006

(54) Isopycnic pharmaceutical formulation

(75) Inventor: Vladislav Nodelman, San Diego, CA (US)

Correspondence Address: AGOURON PHARMACEUTICALS, INC., 10777 SCIENCE CENTER DRIVE, SAN DIEGO, CA, 92121, US

(73) Assignee: Agouron Pharmaceuticals, Inc.

(21) Appl. No.: 10/984,596

(22) Filed: Nov. 8, 2004

(51) Int. Cl. A61K 31/7012; A61K 31/4162

(52) U.S. Cl. 514/53; 514/406

(57) ABSTRACT

The present invention is directed to pharmaceutical compositions comprising an aqueous solution in which the density of the aqueous solution is isopycnic or substantially isopycnic with a pharmaceutical agent. The present invention is also directed to methods for storing such pharmaceutical compositions and formulations, and to the use of such pharmaceutical compositions and formulations to provide benefit to a patient in need of a pharmaceutical agent with limited aqueous solubility.

### BACKGROUND OF THE INVENTION

The present invention is directed to pharmaceutical compositions comprising an aqueous solution in which the density of the aqueous solution is isopycnic or substantially isopycnic with a pharmaceutical agent. The present invention is also directed to methods for storing such pharmaceutical compositions and formulations, and to the use of such pharmaceutical compositions and formulations to provide benefit to a patient in need of a pharmaceutical agent with limited aqueous solubility.

Pharmaceutical agents that have limited solubility in aqueous solutions present a challenge to the formulation scientist. Although an aqueous formulation is often preferred for injectable medicaments, pharmaceutical agents with limited aqueous solubility need to remain suspended in the aqueous solution in order to ensure proper administration of the pharmaceutical agent. In certain instances, flocculation or agglomeration of the poorly soluble pharmaceutical agent may occur during administration by injection.

Furthermore, it is important to be able to formulate various concentrations of the pharmaceutical agent depending on the needs of the patient. Pharmaceutical agents with limited aqueous solubility may not readily be amenable to such needs due to formation of precipitate or sedimentation. In addition, storage of aqueous compositions of pharmaceutical agents with limited aqueous solubility is often limited by the sedimentation of the pharmaceutical agent.

Furthermore, in certain ophthalmic applications, such as eye drops, ointments, and sub-Tenon delivery of a pharmaceutical agent, aqueous formulations are often the preferred form of pharmaceutical composition. In these cases, sedimentation of a poorly soluble pharmaceutical agent is generally disfavored.

Solutions to the aforementioned limitations of pharmaceutical agents with limited aqueous solubility include the use of mixed solvent systems, micelles, highly viscous gels or syrups, and PEGylation of the pharmaceutical agent. Although each of these formulations may find use in certain circumstances with certain pharmaceutical agents, each is also subject to limitations. Mixed solvent systems and micelles may raise toxicity issues, or may cause precipitation of the pharmaceutical agent upon administration to the patient. Highly viscous gels or syrups are difficult to administer by syringe and offer practical difficulties during the manufacturing and filling stages of production. PEGylation may alter the bioavailability or reactivity of the pharmaceutical agent.

Thus, there is a need for improved formulations of pharmaceutical agents with limited aqueous solubility.

### SUMMARY OF THE INVENTION

The present invention is directed to pharmaceutical compositions and formulations comprising an aqueous solution in which the density of the aqueous solution is isopycnic or substantially isopycnic with a pharmaceutical agent. The present invention is also directed to methods for storing such pharmaceutical compositions and formulations, and to the use of such pharmaceutical compositions and formulations to provide benefit to a patient in need of a pharmaceutical agent with limited aqueous solubility.

One embodiment of the present invention is a composition comprising an aqueous phase comprising an aqueous solution of a density modifying agent; and a pharmaceutical agent at least partially in composition in the aqueous phase, wherein the density modifying agent is present in the composition in an amount such that the density of the aqueous phase is essentially the same as the density of the pharmaceutical agent.

One embodiment of the present invention is a composition comprising an aqueous phase comprising an aqueous solution of a density modifying agent; and a pharmaceutical agent at least partially in composition in the aqueous phase, wherein the density modifying agent is present in the composition in an amount such that the density of the aqueous phase is substantially the same as the density of the pharmaceutical agent.

In another embodiment, the amount of the density modifying agent is from 40 to 80 percent by weight, based on the total weight of the aqueous phase. In another embodiment, the amount of the density modifying agent is from 50 to 70 percent by weight, based on the total weight of the aqueous phase. In a further embodiment, the amount of the density modifying agent is from 55 to 65 percent by weight, based on the total weight of the composition.

In one aspect of this embodiment, the density of the pharmaceutical agent is within 5% of the density of the aqueous solution of sucrose. In yet a further aspect the density of the pharmaceutical agent is within 2% of the density of the aqueous solution of sucrose. In yet another further aspect the density of the pharmaceutical agent is within 1% of the density of the aqueous solution of sucrose.

In one aspect of the invention, the density modifying agent is a sweetener, such as sucrose.

In yet another aspect of this embodiment, the pharmaceutical agent is an anti-proliferative agent.

Another embodiment of the present invention is an ophthalmological medicament comprising an aqueous solution of sucrose and a pharmaceutical agent, wherein the amount of sucrose in the aqueous solution of sucrose is from 40-80% of the total weight of the aqueous solution of sucrose and the density of the aqueous solution of sucrose is formulated to have a density substantially identical to the density of the pharmaceutical agent. In one aspect of this embodiment, the pharmaceutical agent is an anti-proliferative agent. In another aspect of this embodiment, the pharmaceutical agent is an inhibitor of a kinase receptor.

Another embodiment of the present invention is an injectible medicament prepared by diluting with water a composition comprising an aqueous solution of sucrose and a pharmaceutical agent, wherein the amount of sucrose in the aqueous solution of sucrose is from 40-80% of the total weight of the aqueous solution of sucrose and the density of the aqueous solution of sucrose is formulated to have a density substantially identical to the density of the pharmaceutical agent. In one aspect of this embodiment, the amount of sucrose in the injectible medicament is from 5-15% of the total weight of the injectable medicament.

Another embodiment of the present invention is a method of preparing a pharmaceutical formulation comprising adding a pharmaceutical agent to a solution comprising water, and adding sufficient sucrose to the solution so that the density of the resulting aqueous solution of sucrose has a density substantially the same to the density of the pharmaceutical agent. The term “substantially the same” means that the density of the solution is such that a stable suspension of a pharmaceutical agent is achieved. Preferably, substantially the same means no more than a 10% difference in the density of the aqueous solution and the density of the pharmaceutical agent. More preferably, the density of the aqueous solution is within 5% of the density of the pharmaceutical agent. More preferably, the density of the aqueous solution is within 2% of the density of the pharmaceutical agent. Most preferably, the density of the aqueous solution is within 1% of the density of the pharmaceutical agent. In one aspect of this embodiment, the method of preparing a pharmaceutical formulation further comprises determining the density of the pharmaceutical agent prior to adding the pharmaceutical agent to the solution comprising water.

Another embodiment of the present invention is a method for the periocular delivery of a pharmaceutical agent comprising sub-Tenon administration to a patient a composition comprising an aqueous solution of sucrose and a pharmaceutical agent, wherein the amount of sucrose in the aqueous solution of sucrose is from 5 to 15% of the total weight of the aqueous solution of sucrose.

Another embodiment of the present invention is a vehicle for the composition of a pharmaceutical agent comprising water and a density modifying agent dissolved therein in which the density of the vehicle is substantially identical to the density of the pharmaceutical agent. The density of the pharmaceutical agent is within 5% of the density of the vehicle.

Another embodiment of the present invention is a method for storing a composition of a pharmaceutical agent in an aqueous solution comprising suspending the pharmaceutical agent in the vehicle in a container.

In one embodiment of the present invention, pharmaceutical agent is a compound of formula 1 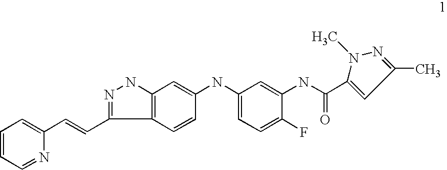
or a pharmaceutically acceptable salt, solvate or hydrate thereof.

The compound 1 can be prepared as described in U.S. Pat. No. 6,531,491, issued Mar. 11, 2003, hereby incorporated by reference in its entirety for all purposes.

In another embodiment, the present invention comprises a composition comprising: an aqueous phase comprising an aqueous solution of a density modifying agent; and a pharmaceutical agent of formula 1 at least partially in composition in the aqueous phase, 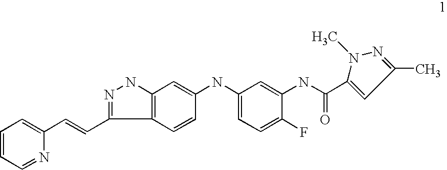
or a pharmaceutically acceptable salt, solvate or hydrate thereof,
wherein the density modifying agent is present in the composition in an amount such that the density of the aqueous phase is essentially the same as the density of the pharmaceutical agent.

In another embodiment, the present invention comprises an aqueous phase comprising an aqueous solution of a density modifying agent in an amount of from 40 to 80 percent by weight, based on the total weight of the aqueous phase; and a pharmaceutical agent of formula 1 at least partially in composition in the aqueous phase, 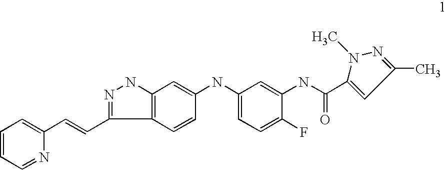
or a pharmaceutically acceptable salt, solvate or hydrate thereof,
wherein then density modifying agent is sucrose, and the density of the aqueous phase is within 10% of the density of the pharmaceutical agent.

In another embodiment, the present invention comprises a composition comprising an aqueous dilution of the aforementioned compositions. Typically, the amount of density modifying agent in the composition is from 5 to 15 percent by weight, based on the total weight of the composition.

In another embodiment, the present invention comprises a method of preparing a pharmaceutical composition, the method comprising forming a composition of a pharmaceutical agent in an aqueous solution, wherein the aqueous solution comprises an amount of a density modifying agent such that the density of the aqueous solution is essentially the same as the density of the pharmaceutical agent.

In another embodiment, the step of forming a composition comprises providing an aqueous solution of the density modifying agent and combining the aqueous solution with the pharmaceutical agent.

In another embodiment, the step of forming a composition comprises combining the pharmaceutical agent, the density modifying agent, and water.

In another embodiment, the present invention comprises a vehicle for the composition of a pharmaceutical agent, comprising an aqueous solution of a density modifying agent, wherein the density of the vehicle is essentially the same as the density of the pharmaceutical agent. In another embodiment, the present invention comprises a method for storing a pharmaceutical composition, comprising suspending the pharmaceutical agent in the vehicle in a container suitable for storage.

In a preferred aspect in any of the above, the density modifying agent is present in an amount from a lower limit of 40 or 50 or 55 to an upper limit of 65 or 70 or 80 percent by weight, based on the total weight of the aqueous phase. Examples of preferred ranges are from 50 to 70 percent by weight, 55 to 65 percent by weight, based on the total weight of the aqueous phase.

In another embodiment, the present invention comprises a composition suitable for administering to a patient comprising an aqueous phase comprising an aqueous solution of a density modifying agent; and a pharmaceutical agent of formula 1 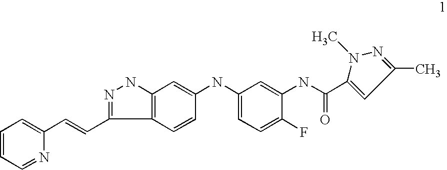
or a pharmaceutically acceptable salt, solvate or hydrate thereof, at least partially in composition in the aqueous phase, wherein the density modifying agent is present in the composition in an amount of from 5 to 15 percent by weight, based on the total weight of the aqueous phase.

In another embodiment, the present invention comprises a composition wherein the amount of density modifying agent is from 7 to 11 percent by weight, based on the total weight of the aqueous phase.

In another embodiment, the present invention comprises a composition wherein the amount of density modifying agent is 8.5 to 9.5 percent by weight, based on the total weight of the aqueous phase.

In another embodiment, the present invention comprises a method of administering to a mammal in need thereof a therapeutically effective amount of the composition suitable for administering to a patient to treat age related macular degeneration, choroidal neovascularization, retinopathy, diabetic retinopathy, vitreoretinopathy and retinopathy of prematurity, retinitis, cytomegalovirus retinitis, uveitis, macular edema, and glaucoma.

In another embodiment, the present invention comprises a method of treating an ophthalmic disease, comprising administering to a mammal in need thereof a therapeutically effective amount of a suitable composition for administering to a patient in combination with at least one of radiation, chemotherapeutic agent, and photodynamic therapy.

In another embodiment, the present invention comprises a method of treating a cancer, comprising administering to a mammal in need thereof a therapeutically effective amount of the suitable composition for administering to a patient in combination with at least one of radiation, chemotherapeutic agent.

In another embodiment, the present invention comprises a method of treating unwanted angiogenesis, comprising administering to a mammal in need thereof a therapeutically effective amount of the suitable composition for administering to a patient in combination with a chemotherapeutic agent.

In a preferred aspect in any of the above, the density modifying agent is a sweetener, for example, sucrose.

In a preferred aspect in any of the above, the density of the aqueous phase is from a lower limit of 1% or 2% or 5% to a higher limit of 10% or 15% of the density of the pharmaceutical agent.

For administration to the eye, a pharmaceutical agent 1 is delivered in a pharmaceutically acceptable ophthalmic vehicle such that the pharmaceutical agent is maintained in contact with the ocular surface for a sufficient time period to allow the pharmaceutical agent to penetrate the cornea and/or sclera and internal regions of the eye, including, for example, the anterior chamber, posterior chamber, vitreous body, aqueous humor, vitreous humor, cornea, iris/cilary, lens, choroid/retina and sclera.

In one embodiment, the present invention comprises a sterile concentrated composition.

In another embodiment, the present invention comprises a sterile aqueous composition suitable for sub-Tenon delivery.

In particular embodiments, pharmaceutical agent 1 is present in a concentrated sterile aqueous composition in an amount ranging from a lower limit of 0.4, 0.5 or 0.6 or 0.7 or 0.8 or 0.9 mg/ml, to an upper limit of 8 or 9 or 10 or 11 or 12 or 13 or 14 or 15 mg/ml, with ranges from any upper limit to any lower limit being contemplated. The concentrated sterile aqueous composition further comprises buffers, such as United States Pharmacopeia (USP) grade dibasic sodium phosphate, anhydrous and monobasic sodium phosphate, monohydrate, commercially available and well known in the art. The buffers are present in amounts to keep the concentrated sterile aqueous composition at a pH of from 4 to 12. The concentrated sterile aqueous composition comprises water, preferably USP grade sterile water for injection. The composition may also further comprise preservatives, which are well known in the art.

The concentrated sterile aqueous composition preferably comprises sucrose. The concentrated sterile aqueous composition comprises sucrose in an amount sufficient to form a stable aqueous composition of pharmaceutical agent represented by formula 1, typically about 40 to 80 weight percent sucrose based on the total weight of the composition. The concentrated sterile aqueous composition comprises sucrose in an amount ranging from a lower limit of 40, 45, 50, 55 or 60 or 65 weight percent, based on the total weight of the composition, to an upper limit of 70 or 75 or 80 weight percent, based on the total weight of the composition, with ranges from any upper limit to any lower limit being contemplated. As illustrated in FIG. 2, different grades of sucrose from different commercial suppliers resulted in a variability in the content uniformities of the concentrated sterile aqueous composition

In another particular embodiment, the pharmaceutical agent 1 is present in a ready to use diluted sterile aqueous composition in an amount ranging from a lower limit of 0.05 or 0.1 or 0.15 or 0.2 or 0.25 or 0.3 mg/ml to an upper limit of 1.2 or 1.3 or 1.4 or 1.5 mg/ml, with ranges from any upper limit to any lower limit being contemplated.

The ready to use diluted sterile aqueous composition comprises the above referenced water, buffers, preservatives, and density modifying agent. Upon dilution with the sterile water to produce the ready to use composition, the sucrose content is present in an amount ranging from a lower limit of 6, 7 or 8 or 9 weight percent, based on the total weight of the composition, to an upper limit of 10 or 11 or 12 weight percent, based on the total weight of the composition, with ranges from any upper limit to any lower limit being contemplated.

The term “isopycnic” means all components in a system are of the same or of substantially the same density.

The term “aqueous solution” means any liquid solution in which the primary liquid component is water. An aqueous solution may contain additional liquid components as well as dissolved gases and solids. Furthermore, not all components of an aqueous solution need be soluble or miscible in water, therefore an aqueous solution may contain immiscible components and suspended (colloidal, micellar, and particulate) components.

The term “density modifying agent” means any material that has high solubility (40-70% by weight) in water, would be considered non-toxic to a typical patient at such high concentrations, is approved for pharmaceutical administration, increases the density of the resulting aqueous solution sufficiently to allow the density of the resulting aqueous solution to be substantially identical to the density of a desired pharmaceutical agent, and has relatively low viscosity. Examples of density modifying agents include polyethylene glycol, and sweeteners.

The phrase “amount of density modifying agent is between about” a certain percentage range “of the total weight of the aqueous solution” means that the weight amount of density modifying agent that is dissolved in the aqueous solution lies within or within standard measurement error of the total weight of the aqueous solution after the density modifying agent has been dissolved therein.

The term “density” encompasses density, relative density, and specific gravity.

The phrase “formulated to be substantially identical” means that a sufficient amount of density modifying agent is dissolved in an aqueous solution so that the density of the resulting aqueous solution is substantially identical to the density of the pharmaceutical agent. Those of skill in the art will recognize that there are multiple ways of achieving such a formulation, including by way of example only, (a) pre-determining the density of the pharmaceutical agent and the density of the resulting aqueous solution; (b) observing the sedimentation rate of the pharmaceutical agent suspended in the resulting aqueous solution; (c) assaying the uniformity of a composition of the pharmaceutical agent in the resulting aqueous solution; and (d) testing the syringeability of a composition of the pharmaceutical agent in the resulting aqueous solution.

The term “sweetener” means a pharmaceutical agent used to impart sweetness to a preparation. Such compounds include, by way of example and without limitation, aspartame, dextrose, glycerin, mannitol, saccharin sodium, sorbitol and sucrose and other materials known to one of ordinary skill in the art.

The phrase “portion of the pharmaceutical agent is suspended in the aqueous solution” means that at least some amount of the pharmaceutical agent is not dissolved in the aqueous solution. That portion of the pharmaceutical agent which is not dissolved is suspended in the aqueous solution, or if sedimentation (partial or otherwise) of the pharmaceutical agent has occurred, then recomposition can readily occur by methods known to those of skill in the art, including shaking, mixing, stirring, sonication, agitation, vortexing, and the like.

The term “ant-proliferative agent” means any pharmaceutical agent that, when administered to a patient, inhibits, halts, prevents, or reverses the unwanted proliferation or growth of cells.

The term “mM” means 1×10-3 Molar (milli molar).

The term “ppm” means part per million.

The term “CI” means confidence interval.

The term “anti-angiogenic agent” means any pharmaceutical agent that, when administered to a patient, inhibits, halts, prevents, or reverses unwanted angiogenesis.

The phrase “inhibits the binding of VEGF to VEGFR-2” means that the agent, when administered to a patient, decreases the amount of VEGF (vascular endothelial growth factor, also known as VEGF-A) that binds to VEGFR-2 (vascular endothelial growth factor receptor 2, also known as flk-1 or KDR or flk-1/KDR).

The phrase “tyrosine kinase receptor inhibitor” means any pharmaceutical agent that, when administered to a patient, decreases the activity of a tyrosine kinase receptor. Such tyrosine kinase receptors include, by way of example only, VEGFR-1, VEGFR-2, PDGF-R, c-Kit, Flt-3, EGFR, CHK1 and MCHR.

The phrase “suitable sub-Tenon administration” means the composition containing the density modifying agent and the pharmaceutical agent is formulated to be administered in the sub-Tenon region of the eye.

The term “diluting” refers to the process of adding additional solvent to a solution in order to decrease the concentration of any agent dissolved therein.

The term “sterile water” refers to water that is suitable for intravenous administration to a patient.

The term “mixing together” refers to the process of causing different substances (here, the pharmaceutical agent and the aqueous solution) to intermingle the components into a more uniform composition. In the present application, a more uniform composition generally refers to a composition of the pharmaceutical agent in the aqueous solution.

The phrase “determining the density” refers to the process of measuring the density of a substance (here, the density of the pharmaceutical agent). Such methods for measuring the density of a substance are well known to those skilled in the art and include, by way of example only, the use of a pycnometer.

The terms “comprising” and “including” are used in an open, non-limiting sense.

Thus, an objective of the invention is to discover potent agents for the treatment of ophthalmic diseases, such as age related macular degeneration (ARMD), choroidal neovascularization (CNV), retinopathies (e.g., diabetic retinopathy, vitreoretinopathy, retinopathy of prematurity), retinitis (e.g., cytomegalovirus (CMV) retinitis), uveitis, macular edema, and glaucoma.

Another objective of the present invention is to discover potent formulations of inhibitors of protein kinases.

Another objective of the invention is to discover effective formulations of kinase inhibitors having a strong and selective affinity for one or more particular kinases.

These and other objectives of the invention, which will become apparent from the following description, have been achieved by the discovery of pharmaceutical agents, pharmaceutically acceptable prodrugs, pharmaceutically active metabolites, and pharmaceutically acceptable salts thereof (such compounds, prodrugs, metabolites and salts are collectively referred to as “agents”) described below, which modulate and/or inhibit the activity of protein kinases. Pharmaceutical compositions containing such agents are useful in treating diseases mediated by kinase activity, such as cancer, as well as other disease states associated with unwanted angiogenesis and/or cellular proliferation, such as diabetic retinopathy, neovascular glaucoma, rheumatoid arthritis, and psoriasis. Further, the agents have advantageous properties relating to the modulation and/or inhibition of the kinase activity associated with VEGF-R, FGF-R, CDK complexes, CHK1, LCK, TEK, FAK, and/or phosphorylase kinase.

The invention also relates to a method of modulating and/or inhibiting the kinase activity of VEGF-R, FGF-R, a CDK complex, CHK1, LCK, TEK, FAK, and/or phosphorylase kinase by administering a pharmaceutical agent 1 or a pharmaceutically acceptable prodrug, pharmaceutically active metabolite, or pharmaceutically acceptable salt thereof.

The inventive pharmaceutical agents may be used advantageously in combination with other known therapeutic agents. For example, pharmaceutical agent 1, which possesses antiangiogenic activity may be co-administered with cytotoxic chemotherapeutic agents, such as taxol, taxotere, vinblastine, cis-platin, doxorubicin, adriamycin, and the like, to produce an enhanced antitumor effect. Additive or synergistic enhancement of therapeutic effect may also be obtained by co-administration of pharmaceutical agents 1 which possess antiangiogenic activity with other antiangiogenic agents, such as combretastatin A-4, endostatin, prinomastat, celecoxib, rofocoxib, EMD121974, IM862, anti-VEGF monoclonal antibodies, and anti-KDR monoclonal antibodies.

Further, the present invention may be used in combination with other ophthalmic treatments, such as photodynamic therapy (PDT), photocoagulation, and anti-angiogenesis inhibitors.

The invention also relates to pharmaceutical compositions, each comprising an effective amount of an agent selected from pharmaceutical agent 1 and pharmaceutically acceptable salts, pharmaceutically active metabolites, and pharmaceutically acceptable prodrugs thereof; and a pharmaceutically acceptable carrier or vehicle for such agent. The invention further provides methods of treating ophthalmic diseases/conditions and cancer as well as other disease states associated with unwanted angiogenesis and/or cellular proliferation, comprising administering effective amounts of such an agent to a patient in need of such treatment.

In another embodiment, the invention provides a method of treating cancer in a mammal, including a human, the method comprising administering to the mammal a therapeutically effective amount of any of the pharmaceutical compositions of the invention.

In another embodiment, the invention provides a method of treating cancer in a mammal, the method comprising administering to the mammal, including a human, any of the capsules of the invention.

In a particular aspect of any of the preceding method embodiments, the method further comprises administering one or more anti-tumor agents, anti-angiogenesis agents, signal transduction inhibitors, or antiproliferative agents.

The invention also relates to a method for the treatment of abnormal cell growth in a mammal, including a human, comprising administering to said mammal an amount of a compound of the formula 1, as defined above, or a pharmaceutically acceptable salt, solvate or prodrug thereof, that is effective in treating abnormal cell growth. In one embodiment of this method, the abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said method, said abnormal cell growth is a benign proliferative disease, including, but not limited to, psoriasis, benign prostatic hypertrophy or restinosis.

This invention also relates to a method for the treatment of abnormal cell growth in a mammal which comprises administering to said mammal an amount of a pharmaceutical agent 1, or a pharmaceutically acceptable salt, solvate or prodrug thereof, that is effective in treating abnormal cell growth in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

This invention also relates to a pharmaceutical composition for the treatment of abnormal cell growth in a mammal, including a human, comprising an amount of a pharmaceutical agent 1, as defined above, or a pharmaceutically acceptable salt, solvate or prodrug thereof, that is effective in treating abnormal cell growth, and a pharmaceutically acceptable carrier. In one embodiment of said composition, said abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said pharmaceutical composition, said abnormal cell growth is a benign proliferative disease, including, but not limited to, psoriasis, benign prostatic hypertrophy or restinosis.

The invention also relates to a pharmaceutical composition for the treatment of abnormal cell growth in a mammal, including a human, which comprises an amount of a pharmaceutical agent 1, as defined above, or a pharmaceutically acceptable salt, solvate or prodrug thereof, that is effective in treating abnormal cell growth in combination with a pharmaceutically acceptable carrier and an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

This invention also relates to a method for the treatment of a disorder associated with angiogenesis in a mammal, including a human, comprising administering to said mammal an amount of a pharmaceutical agent 1, as defined above, or a pharmaceutically acceptable salt, solvate or prodrug thereof, that is effective in treating said disorder. Such disorders include cancerous tumors such as melanoma; ocular disorders such as age-related macular degeneration, presumed ocular histoplasmosis syndrome, and retinal neovascularization from proliferative diabetic retinopathy; rheumatoid arthritis; bone loss disorders such as osteoporosis, Paget's disease, humoral hypercalcemia of malignancy, hypercalcemia from tumors metastatic to bone, and osteoporosis induced by glucocorticoid treatment; coronary restenosis; and certain microbial infections including those associated with microbial pathogens selected from adenovirus, hantaviruses, Borrelia burgdorferi, Yersinia spp., Bordetella pertussis, and group A Streptococcus.

This invention also relates to a method of (and to a pharmaceutical composition for) treating abnormal cell growth in a mammal which comprise an amount of a pharmaceutical agent 1, or a pharmaceutically acceptable salt, solvate or prodrug thereof, and an amount of one or more substances selected from anti-angiogenesis agents, signal transduction inhibitors, and antiproliferative agents, which amounts are together effective in treating said abnormal cell growth.

Within other embodiments of the invention, anti-angiogenic compositions may be implanted by injection of the composition into the region of the anterior chamber angle. This provides a sustained localized increase of anti-angiogenic factor, and prevents blood vessel growth into the area. Implanted or injected anti-angiogenic compositions which are placed between the advancing capillaries of the iris and the anterior chamber angle can “defend” the open angle from neovascularization. As capillaries will not grow within a significant radius of the anti-angiogenic composition, patency of the angle could be maintained. Within other embodiments, the anti-angiogenic composition may also be placed in any location such that the anti-angiogenic factor is continuously released into the aqueous humor. This would increase the anti-angiogenic factor concentration within the humor, which in turn bathes the surface of the iris and its abnormal capillaries, thereby providing another mechanism by which to deliver the medication. These therapeutic modalities may also be useful prophylactically and in combination with existing treatments.

The formulas are intended to cover solvated as well as unsolvated forms of the identified structures. For example, pharmaceutical agent 1 includes compounds of the indicated structure in both hydrated and non-hydrated forms. Other examples of solvates include the structures in combination with isopropanol, ethanol, methanol, dimethyl sulfoxide (DMSO), ethyl acetate, acetic acid, or ethanolamine.

In addition to pharmaceutical agent 1, the invention includes pharmaceutically acceptable prodrugs, pharmaceutically active metabolites, and pharmaceutically acceptable salts of such pharmaceutical agents.

“A pharmaceutically acceptable prodrug” is a compound that may be converted under physiological conditions or by solvolysis to the specified compound or to a pharmaceutically acceptable salt of such compound.

“A pharmaceutically active metabolite” is intended to mean a pharmacologically active product produced through metabolism in the body of a specified compound or salt thereof. Metabolites of a compound may be identified using routine techniques known in the art and their activities determined using tests such as those described herein.

Prodrugs and active metabolites of a compound may be identified using routine techniques known in the art. See, e.g., Bertolini, G., et al., J. Med. Chem., 40, 2011-2016 (1997); Shan, D., et al., J. Pharm. Sci., 86 (7), 765-767; Bagshawe K., Drug Dev. Res., 34, 220-230 (1995); Bodor, N., Advances in Drug Res., 13, 224-331 (1984); Bundgaard, H., Design of Prodrugs (Elsevier Press 1985); and Larsen, I. K., Design and Application of Prodrugs, Drug Design and Development (Krogsgaard-Larsen et al., eds., Harwood Academic Publishers, 1991).

“A pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

In the case of agents that are solids, it is understood by those skilled in the art that the inventive compounds and salts may exist in different crystal or polymorphic forms, all of which are intended to be within the scope of the present invention and specified formulas.

Anti-angiogenesis agents, such as MMP-2 (matrix-metalloprotienase 2) inhibitors, MMP-9 (matrix-metalloprotienase 9) inhibitors, and COX-II (cyclooxygenase II) inhibitors, can be used in conjunction with a pharmaceutical agent 1 in the methods and pharmaceutical compositions described herein. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are herein incorporated by reference in their entirety. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Some specific examples of MMP inhibitors useful in combination with the compounds of the present invention are AG-3340, RO 32-3555, RS 13-0830, and the compounds recited in the following list:
3-[[4-(4-fluoro-phenoxy)-benzenesulfonyl]-(1-hydroxycarbamoyl-cyclopentyl)-amino]-propionic acid;
3-exo-3-[4-(4-fluoro-phenoxy)-benzenesulfonylamino]-8-oxa-bicyclo[3.2.1]octane-3-carboxylic acid hydroxyamide;
(2R,3R) 1-[4-(2-chloro-4-fluoro-benzyloxy)-benzenesulfonyl]-3-hydroxy-3-methyl-piperidine-2-carboxylic acid hydroxyamide;
4-[4-(4-fluoro-phenoxy)-benzenesulfonylamino]-tetrahydro-pyran-4-carboxylic acid hydroxyamide;
3-[[4-(4-fluoro-phenoxy)-benzenesulfonyl]-(1-hydroxycarbamoyl-cyclobutyl)-amino]-propionic acid;
4-[4-(4-chloro-phenoxy)-benzenesulfonylamino]-tetrahydro-pyran-4-carboxylic acid hydroxyamide;
3-[4-(4-chloro-phenoxy)-benzenesulfonylamino]-tetrahydro-pyran-3-carboxylic acid hydroxyamide;
(2R,3R) 1-[4-(4-fluoro-2-methyl-benzyloxy)-benzenesulfonyl]-3-hydroxy-3-methyl-piperidine-2-carboxylic acid hydroxyamide;
3-[[4-(4-fluoro-phenoxy)-benzenesulfonyl]-(1-hydroxycarbamoyl-1-methyl-ethyl)-amino]-propionic acid;
3-[[4-(4-fluoro-phenoxy)-benzenesulfonyl]-(4-hydroxycarbamoyl-tetrahydro-pyran-4-yl)-amino]-propionic acid;
3-exo-3-[4-(4-chloro-phenoxy)-benzenesulfonylamino]-8-oxa-bicyclo[3.2.1]octane-3-carboxylic acid hydroxyamide;
3-endo-3-[4-(4-fluoro-phenoxy)-benzenesulfonylamino]-8-oxa-bicyclo[3.2.1]octane-3-carboxylic acid hydroxyamide; and
3-[4-(4-fluoro-phenoxy)-benzenesulfonylamino]-tetrahydro-furan-3-carboxylic acid hydroxyamide;
   and pharmaceutically acceptable salts, solvates and prodrugs of said compounds.

The pharmaceutical agents of formula 1, and the pharmaceutically acceptable salts, solvates and prodrugs thereof, can also be used in combination with signal transduction inhibitors, such as agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, for example, HERCEPTIN™ (Genentech, Inc. of South San Francisco, Calif., USA).

EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998). EGFR-inhibiting agents include, but are not limited to, the monoclonal antibodies C225 and anti-EGFR 22Mab (ImClone Systems Incorporated of New York, N.Y., USA), the compounds ZD-1839 (AstraZeneca), BIBX-1382 (Boehringer Ingelheim), MDX-447 (Medarex Inc. of Annandale, N.J., USA), and OLX-103 (Merck & Co. of Whitehouse Station, N.J., USA), VRCTC-310 (Ventech Research) and EGF fusion toxin (Seragen Inc. of Hopkinton, Mass.).

VEGF inhibitors, for example SU-5416 and SU-6668 (Sugen Inc. of South San Francisco, Calif., USA), can also be combined with a pharmaceutical agent of formula 1. VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety. Other examples of some specific VEGF inhibitors are IM862 (Cytran Inc. of Kirkland, Wash., USA); anti-VEGF monoclonal antibody of Genentech, Inc. of South San Francisco, Calif.; and angiozyme, a synthetic ribozyme from Ribozyme (Boulder, Colo.) and Chiron (Emeryville, Calif.).

ErbB2 receptor inhibitors, such as GW-282974 (Glaxo Wellcome plc), and the monoclonal antibodies AR-209 (Aronex Pharmaceuticals Inc. of The Woodlands, Tex., USA) and 2B-1 (Chiron), may be administered in combination with a pharmaceutical agent 1. Such erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are herein incorporated by reference in their entirety.

Other antiproliferative agents that may be used with the compounds of the present invention include inhibitors of the enzyme farnesyl protein transferase and inhibitors of the receptor tyrosine kinase PDGFr, including the compounds disclosed and claimed in the following U.S. patent applications Ser. No. 09/221,946 (filed Dec. 28, 1998); Ser. No. 09/454,058 (filed Dec. 2, 1999); Ser. No. 09/501,163 (filed Feb. 9, 2000); Ser. No. 09/539,930 (filed Mar. 31, 2000); Ser. No. 09/202,796 (filed May 22, 1997); Ser. No. 09/384,339 (filed Aug. 26, 1999); and Ser. No. 09/383,755 (filed Aug. 26, 1999); and the compounds disclosed and claimed in the following U.S. provisional patent applications Nos. 60/168,207 (filed Nov. 30, 1999); 60/170,119 (filed Dec. 10, 1999); 60/177,718 (filed Jan. 21, 2000); 60/168,217 (filed Nov. 30, 1999), and 60/200,834 (filed May 1, 2000). Each of the foregoing patent applications and provisional patent applications is herein incorporated by reference in their entirety.

A compound of formula 1 may also be used with other agents useful in treating abnormal cell growth or cancer, including, but not limited to, agents capable of enhancing anti-tumor immune responses, such as CTLA4 (cytotoxic lymphocite antigen 4) antibodies, and other agents capable of blocking CTLA4; and anti-proliferative agents such as other farnesyl protein transferase inhibitors. Specific CTLA4 antibodies that can be used in the present invention include those described in U.S. Provisional Application 60/113,647 (filed Dec. 23, 1998) which is herein incorporated by reference in its entirety.

### BRIEF DESCRIPTION OF THE DRAWINGS

FIG. 1 illustrates the relationship between the buffer strength, pH and impurity content.

FIG. 2 illustrates the sucrose effect on content uniformity in the ready to use (RTU) composition.

FIG. 3 illustrates the effect of pharmaceutical agent 1 concentration in the (RTU) composition on content uniformity (CU).

FIG. 4 illustrates the effect on content uniformity (CU) of pharmaceutical agent 1 inter-lot variability.

FIG. 5 illustrates the dilution process and dosing.

### DETAILED DESCRIPTION OF INVENTION AND PREFERRED EMBODIMENTS

Several diseases and conditions of the posterior segment of the eye threaten vision. Age related macular degeneration (ARMD), choroidal neovascularization (CNV), retinopathies (e.g., diabetic retinopathy, vitreoretinopathy, retinopathy of prematurity), retinitis (e.g., cytomegalovirus (CMV) retinitis), uveitis, macular edema, and glaucoma are several examples.

Several methods of treatment are currently being developed, such as photocoagulation, photodynamic therapy, and the use of angiogenesis inhibitors.

Photocoagulation uses high intensity laser illumination to create tissue burns and coagulates the new vessels, e.g., in diabetic retinopathy, they use “pan laser photocoagulation” to treat. (ie, they just burn small holes in a regular pattern across the retina). However, photocoagulation can be harmful to the retina and is impractical when the CNV is in proximity of the fovea. Furthermore, photocoagulation often results in recurrent CNV over time.

Photodynamic therapy (PDT) uses dyes injected intravenous (IV) to cause vascular occlusion by a photochemical reaction with a laser shone in the eye (causes less damage because just the vessels are targeted). (eg verteporfin is a photosensitizer-type PDT for AMD).

Oral administration of anti-angiogenic compounds is also being tested as a systemic treatment for ARMD. However, due to drug-specific metabolic restrictions, systemic administration usually provides sub-therapeutic drug levels to the eye. Therefore, to achieve effective intraocular drug concentrations, either an unacceptably high dose or repetitive conventional doses are required. Various implants have also been developed for delivery of anti-angiogenic compounds locally to the eye. Examples of such implants are disclosed in U.S. Pat. No. 5,824,072 to Wong, U.S. Pat. No. 5,476,511 to Gwon, et al., and U.S. Pat. No. 5,773,019 to Ashton, et al. Neovascular Diseases of the Eye.

As noted above, the present invention also provides methods for treating neovascular diseases of the eye, including for example, corneal neovascularization, neovascular glaucoma, proliferative diabetic retinopathy, retrolental fibroblasia and macular degeneration.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

Therapeutically effective amounts of the agents of the invention may be used to treat diseases mediated by modulation or regulation of protein kinases. An “effective amount” is intended to mean that amount of an agent that, when administered to a mammal in need of such treatment, is sufficient to effect treatment for a disease mediated by the activity of one or more protein kinases, such as tyrosine kinases. Thus, e.g., a therapeutically effective amount of a compound of the Formula I, salt, active metabolite or prodrug thereof is a quantity sufficient to modulate, regulate, or inhibit the activity of one or more protein kinases such that a disease condition which is mediated by that activity is reduced or alleviated.

The amount of a given agent that will correspond to such an amount will vary depending upon factors such as the particular compound, disease condition and its severity, the identity (e.g., weight) of the mammal in need of treatment, but can nevertheless be routinely determined by one skilled in the art. “Treating” is intended to mean at least the mitigation of a disease condition in a mammal, such as a human, that is affected, at least in part, by the activity of one or more protein kinases, such as tyrosine kinases, and includes: preventing the disease condition from occurring in a mammal, particularly when the mammal is found to be predisposed to having the disease condition but has not yet been diagnosed as having it; modulating and/or inhibiting the disease condition; and/or alleviating the disease condition.

The active agents of the invention may be formulated into pharmaceutical compositions as described below. Pharmaceutical compositions of this invention comprise an effective modulating, regulating, or inhibiting amount of a pharmaceutical agent of formula 1 and an inert, pharmaceutically acceptable carrier or diluent. In one embodiment of the pharmaceutical compositions, efficacious levels of the inventive agents are provided so as to provide therapeutic benefits involving modulation of protein kinases. By “efficacious levels” is meant levels in which the effects of protein kinases are, at a minimum, regulated. These compositions are prepared in unit-dosage form appropriate for the mode of administration.

It will be appreciated that the actual dosages of the agents used in the compositions of this invention will vary according to the particular complex being used, the particular composition formulated, the mode of administration and the particular site, host and disease being treated. Optimal dosages for a given set of conditions can be ascertained by those skilled in the art using conventional dosage-determination tests in view of the experimental data for an agent.

Proper formulation is dependent upon the route of administration chosen. For injection, the agents of the invention may be formulated into aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

An inventive agent is administered in conventional dosage form prepared by combining a therapeutically effective amount of an agent (e.g., a pharmaceutical agent of Formula I) as an active ingredient with appropriate pharmaceutical carriers or diluents according to conventional procedures. These procedures may involve mixing, granulating and compressing or dissolving the ingredients as appropriate to the desired preparation.

A pharmaceutical agent of the invention may also be injected directly into the vitreous humor or aqueous humor.

Further, a pharmaceutical agent may be also be administered by acceptable methods, such as subtebnon and/or subconjunctival injections. As is well known in the ophthalmic art, the macula is comprised primarily of retinal cones and is the region of maximum visual acuity in the retina. A Tenon's capsule or Tenon's membrane is disposed on the sclera. A conjunctiva covers a short area of the globe of the eye posterior to the limbus (the bulbar conjunctiva) and folds up (the upper cul-de-sac) or down (the lower cul-de-sac) to cover the inner areas of the upper eyelid and lower eyelid, respectively. The conjunctiva is disposed on top of Tenon's capsule.

The sclera and Tenon's capsule define the exterior surface of the globe of the eye. For treatment of ARMD, CNV, retinopathies, retinitis, uveitis, cystoid macular edema (CME), glaucoma, and other diseases or conditions of the posterior segment of the eye, it is preferable to dispose a depot of a specific quantity of an ophthalmically acceptable pharmaceutically active agent directly on the outer surface of the sclera and below Tenon's capsule. In addition, in cases of ARMD and CME it is most preferable to dispose the depot directly on the outer surface of the sclera, below Tenon's capsule, and generally above the macula. In a study using New Zealand White rabbits, a drug depot of 4,9(11)-Pregnadien-17.alpha.,21-diol-3,20-dione-21-acetate, an angiostatic steroid available from Steraloids, Inc. of Wilton, N.H., was disposed directly on the outer surface of the sclera, below the Tenon's capsule, and slightly posterior of the equator of the rabbit eyes. Such a drug depot resulted in a concentration of the angiostatic steroid, averaged over the entire retina and measured the day after the injection, about ten times greater than a similar concentration delivered by a depot located below the conjunctiva but above the Tenon's capsule of the rabbit eyes. Given the fact that the Tenon's capsule of a New Zealand White rabbit is very thin, these beneficial results are highly unexpected. It is important to note that Tenon's capsule of the human eye is also very thin. 4,9(11)-Pregnadien-17.alpha.,21-diol-3,20-dione-21-acetate, and the related compound 4,9(11)-Pregnadien-17.alpha.,21-diol-3,20-dione, are more fully described in U.S. Pat. Nos. 5,770,592 and 5,679,666, which are incorporated herein in their entireties by reference for all purposes.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Some of the compounds of the invention may be provided as salts with pharmaceutically compatible counter ions. Pharmaceutically compatible salts may be formed with many acids, including hydrochloric, sulfuric, acetic, lactic, tartaric, malic, succinic, etc. Salts tend to be more soluble in aqueous or other protonic solvents than are the corresponding free-base forms.

In the examples described below, unless otherwise indicated all temperatures are set forth in degrees Celsius and all parts and percentages are by weight. Reagents were purchased from commercial suppliers such as Aldrich Chemical Company or Lancaster Synthesis Ltd. and were used without further purification unless otherwise indicated.

### EXAMPLES

To make an isopycnic composition of a drug substance in sucrose, the drug particles are suspended in a sucrose solution in which the concentration of sucrose is sufficient to form a stable composition of the drug particles, typically from 40-80%, and is adjusted to approximate the density of the sucrose solution to the density of the drug substance. For example, a 40% sucrose solution has a density of 1.17 g/mL and a 70% sucrose solution has a density of 1.34 g/mL. An isopycnic composition in which the drug remains suspended indefinitely can be made by adjusting the sucrose solution density to be substantially the same as the drug substance density.

To minimize sedimentation during manufacturing and filling, conventional vehicles are made to be highly viscous. This minimizes drug settling, but also reduces the flowability of the drug product, which makes it more difficult to mix and fill. In these conventional vehicles, sedimentation is minimized, but not eliminated. After sufficiently long storage, the drug substance sediments and must be re-suspended. Re-composition efficiency is inversely proportional to the viscosity of the vehicle, and therefore a significant amount of energy must be expanded to re-suspend drug substance in a viscous vehicle. This is not the case for an isopycnic vehicle for two reasons. First, sedimentation is eliminated by the density of the drug substance and the vehicle, and therefore re-composition is unnecessary. Second, the viscosity of the vehicle is significantly less than that of traditional viscous vehicle, which facilitates mixing and filling of the drug product.

The following examples illustrate several different formulations.

In one embodiment, the present invention comprises a sterile aqueous concentrated composition of a pharmaceutical agent 1. For example, the present composition comprises the free base of the pharmaceutical agent 1

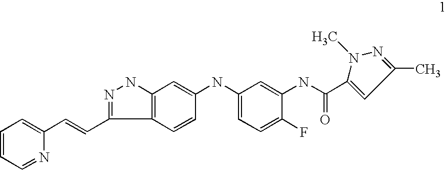

a crystalline monohydrate with approximately less than 10% moisture content, more preferably less than 5%, and most preferably less than or equal to 3.7% moisture content.

The pharmaceutical agent 1 is light insensitive and stable at room temperature. The pharmaceutical agent is stable following gamma irradiation at 25 kGy and is stable following e-beam irradiation at 25 kGY. The particle size distribution, calculated by means well known in the art, following jet-milling (in microns) is as follows:

d₁₀=0.5; d₅₀=1.5; and d₉₀=3.5; wherein for example, d₅₀ represents 50% of the pharmaceutical agent is 1.5 microns or less in size.

The dosage form should be terminally sterilizable, stable at room temperature for approximately more than 24 months, and comprise less than 100 ppm of 5-HMF degradation impurities after autoclaving.

The compositions of the invention may be manufactured in manners generally known for preparing pharmaceutical compositions, e.g., using conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active pharmaceutical agents into preparations which can be used pharmaceutically.

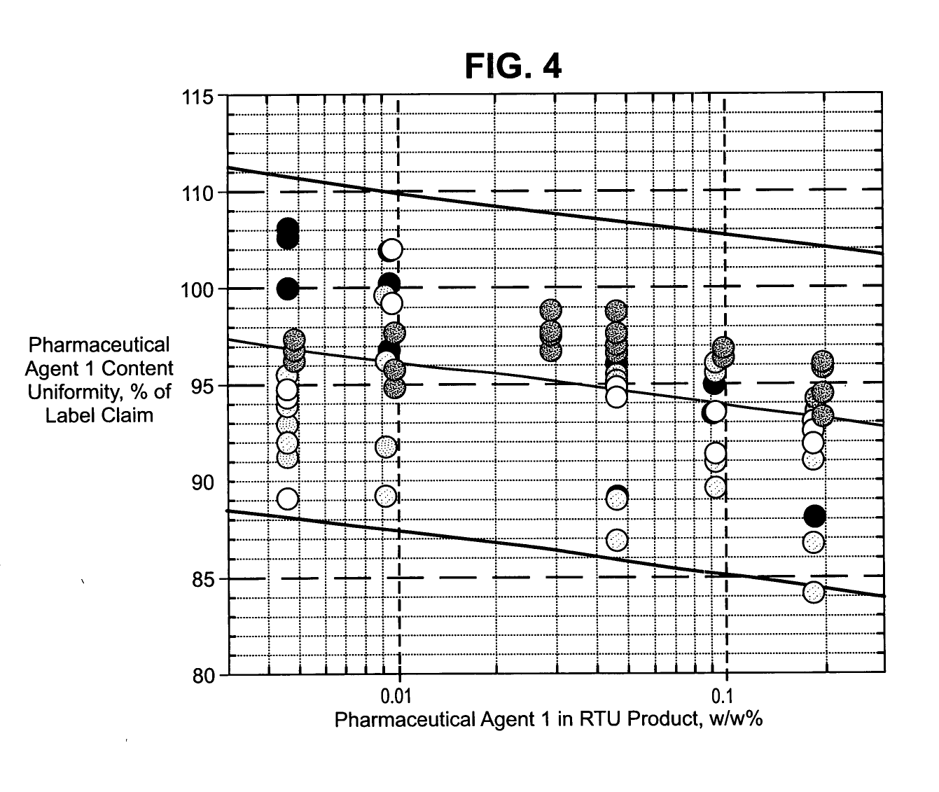

The clinical manufacturing process, as illustrated in FIG. 4, comprises hydrating the vehicle at room temperature comprising high shear mixing. The vehicle undergoes high shear mixing for approximately thirty minutes or less. The in process control (IPC) is performed by measuring the density, pH, and the bioburden.

The pharmaceutical agent 1 can be Milled in composition: 1 hour at 7000-8000 rpm for a 14 L batch with a 2 inch mixing head and an addition rate of 2 grams powder per minute. This is heavily dependent upon the batch size.

The density is measured by well known methods in the art to confirm the sucrose concentration. The pH measurement confirms the buffer additions and the bioburden measures the biological contamination. The vehicle is then filtered through at least one in-series micron filters. The drug substance and the vehicle are then high shear mixed together at room temperature for approximately 30 minutes. Before vials are filled, the mixture undergoes another in process control inspection (IPC), comprising density measurements, pH measurements, bioburden, content uniformity measurements.

The mixture is then packaged in vials, preferably such that headspace is minimized. Stoppers, preferably West 4432/50 B2-42 coated, are used to stop the stoppers and then the vials and stoppers are sealed, preferably with West 20 mm flip off/tear off aluminum. The sealed composition in the vials are terminally sterilized, preferably at 121° C. for approximately 15 minutes.

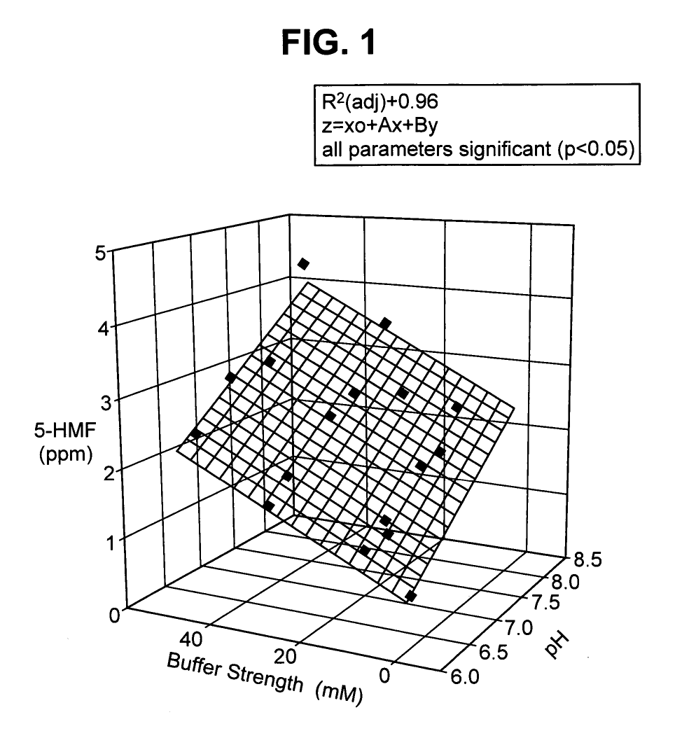

The autoclaving has some degradation effect on the sucrose composition. See FIG. 1. The sucrose may degrade and form 5-HMF (hydroxymethyl-2-furfuraldehyde). A pH shift (7.1 to 6.8 in concentrated composition) is noted along with increased discoloration of the composition in the vial. The PH and buffer strength seem to have an effect on formation of 5-HMF. Preferably, if the pH is kept below 10, lower amounts of 5-HMF are produced during the autoclaving. It is not necessary, but it is preferred that the concentration of 5-HMF not be present in more than 100 ppm. More preferably, it is preferred that the concentration of 5-HMF be no more than 80 ppm. Also, in FIG. 1, the block of information where R² (adj)=0.096 means regression coefficient adjusted for the number of data points. Where z=xo+Ax+By means regression equation; z is a 5-HMF concentration, x is buffer strength, and y is pH. All parameters significant (p<0.05) means parameters xo, A, and B in the regression equation are constants of regression and are significantly different from zero, probability of incorrectly concluding this is less than 5%.

### Dilution

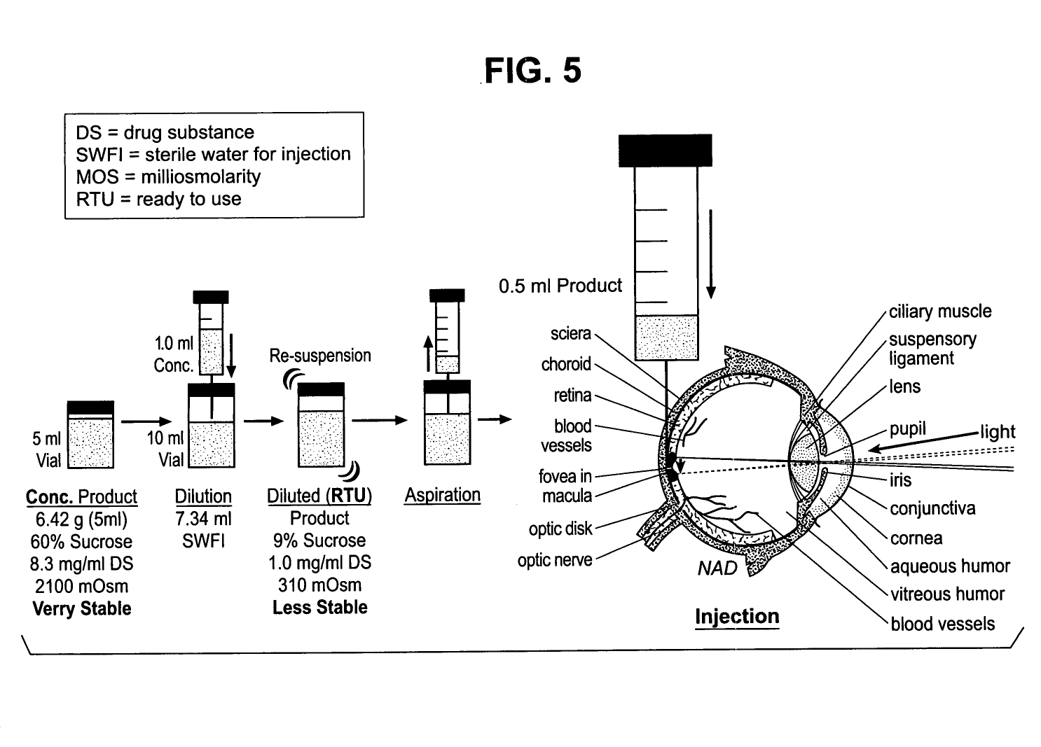

In preparation for administration to the patient, a predetermined amount of the sterile aqueous concentrated composition is diluted into a predetermined amount of sterile water in a separate container. The diluted, ready to use composition is then agitated. A new predetermined amount of composition is removed from the container and administered to the sub-Tenon area of the patient, typically using a syringe with a cannula. As illustrated in FIG. 5, the sucrose concentration drops to approximately 9% weight.

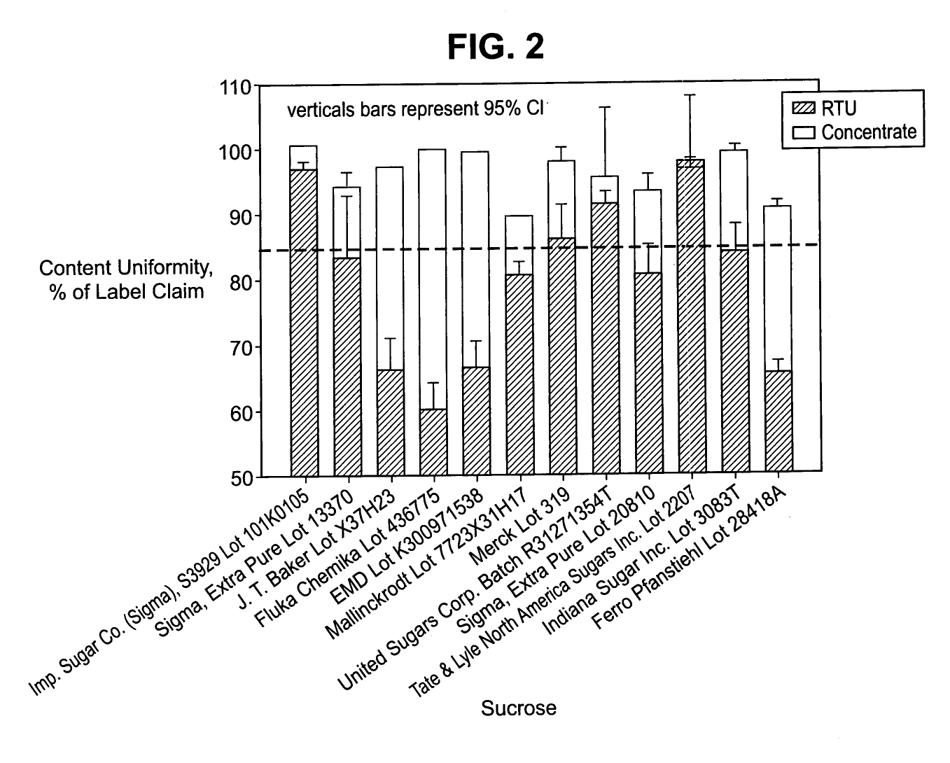

As illustrated in FIG. 2, sucrose received from different vendors produced different results. The sucrose received from Sigma, S3929 Lot 101K0105, Merck Lot 319, United Sugars Corp, Batch R31271354T, and Tate & Lyle North America Sugars Inc Lot 2207 exhibited the best content uniformity. The vertical bars represent 95% Confidence Interval.

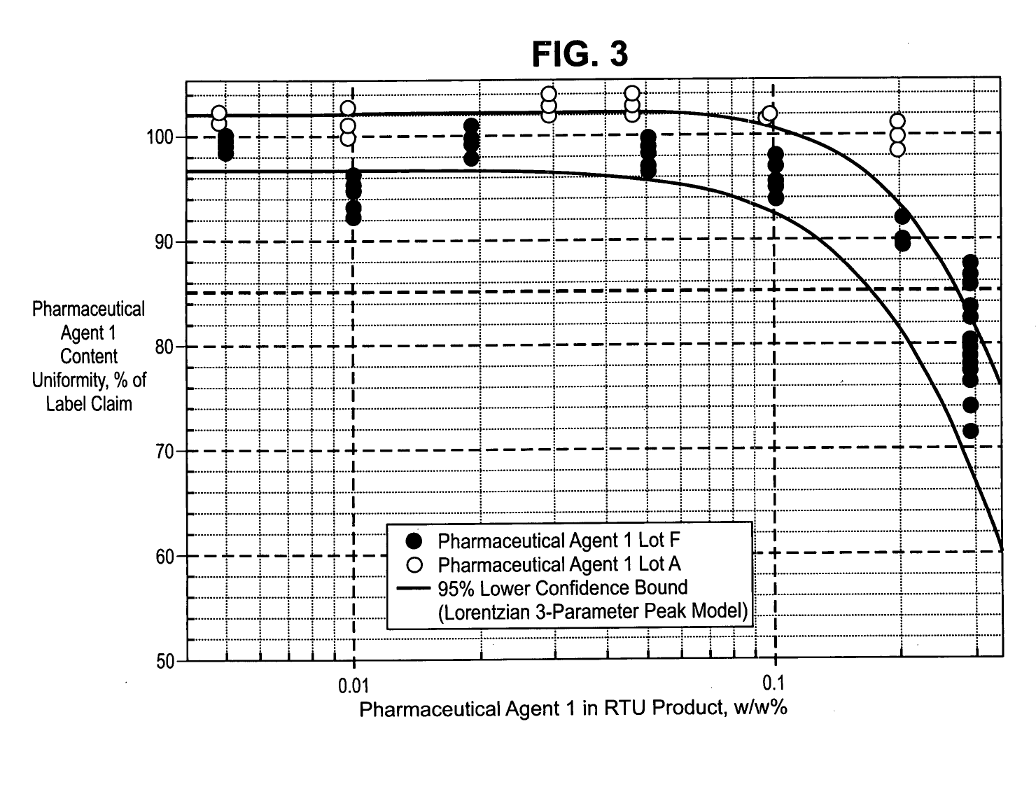

As shown in FIG. 3, the drug substance Lot also exhibited an effect on the content uniformity on the ready to use composition as evidenced in Lots A though E.

As shown in FIG. 4, the various lots of the drug substance had an effect on the content uniformity.

The variability of the drug substance lots and commercial sucrose lots led to the decrease in the assay of the concentrate below 90% after one week. The storage stability is also independent of the storage temperature. Possible degradation may be due to caking of the drug substance at the stopper/glass interface of the vial when the vials were not stored in an upright position.

The composition may be delivered to the sub-Tenon capsule of the eye. Delivery via a cannula is preferred.

### Example 1

Dosage Form Description: Clear, light amber solution. The density of the solution is approximately 1.284 g/mL and the viscosity of the solution is approximately 0.5 Poise at 25° C. In the chart below, “NF” means National Formulary, “USP” means United States Pharmacopeia, and “House” means Pfizer La Jolla Labs.

Quantitative Composition:

| | | Theoretical Quantity | | |
|---|---|---|---|---|
| Ingredient | Grade | (mass/gram) | | % w/w |
| Dibasic Sodium | USP | 4.48 | mg/g | 0.448 |
| Phosphate, Anhydrous | | | | |
| Monobasic Sodium | USP | 1.02 | mg/g | 0.102 |
| Phosphate, Monohydrate | | | | |
| Sucrose | NF | 600.00 | mg/g | 60.000 |
| Water for Injection | USP | 394.50 | mg/g | 39.450 |
| Total Target Weight | | 1 | g | 100.00% |

Manufacturing Process:

The manufacturing process is carried out at room temperature. The phosphates are combined in the water, followed by the sucrose. The resulting solution is sterile filtered and 6.42 g is filled into a 5 mL vial (Type I glass, USP). The vial is closed with a rubber stopper, crimped with an aluminum crimp seal, and terminally sterilized. Prior to use, the solution is rendered isotonic by dilution with Sterile Water for Injection.

### Example 2

Composition for Injection, 10.0 mg/mL

Dosage Form Type: Concentrated composition to be diluted to yield a 1.2 mg/mL composition
Dosage Form Description: Opaque, off-white-light yellow composition. The density of the composition is approximately 1.284 g/mL and the viscosity of the composition is approximately 0.5 Poise at 25° C. For sub-Tenon use only.

Quantitative Composition:

| | | Theoretical Quantity | | |
|---|---|---|---|---|
| Ingredient | Grade | (mass/gram) | | % w/w |
| Pharmaceutical agent | House | 7.787 | mg/g | 0.7787 |
| of formula 1, micronized* | | | | |
| Dibasic Sodium | USP | 4.445 | mg/g | .04445 |
| Phosphate, Anhydrous | | | | |
| Monobasic Sodium | USP | 1.012 | mg/g | 0.1012 |
| Phosphate, Monohydrate | | | | |
| Sucrose | NF | 595.328 | mg/g | 59.5328 |
| Water for Injection | USP | 391.428 | mg/g | 39.1428 |
| Total Target Weight | | 1 | g | 100.00% |

*adjusted for purity and moisture content

Manufacturing Process:

The manufacturing process is carried out at room temperature. The sucrose vehicle is prepared by sequentially combining the phosphates and sucrose in water, followed by sterile filtration of the resulting solution. The vehicle is then mixed with 0.7787% AG-013958 drug substance by weight to yield a 10.002 mg/mL composition. Into a 5 mL vial (Type 1 glass, USP) is added 6.42 g of the composition. The vial is closed with a rubber stopper, crimped with an aluminum crimp seal, and terminally sterilized. Prior to use, the composition is diluted with sterile water for injection (USP or equivalent) or diluent to yield a 1.2 mg/mL pharmaceutical agent represented by formula 1 isotonic composition.

### Example 3

Concentrated Composition for Sub-Tenon Injection, 8.3 mg/ml (to be diluted to yield a 1.0 mg/ml composition)

| | | Theoretical Quantity | | |
|---|---|---|---|---|
| Ingredient | Grade | (mass/gram) | | % w/w |
| Pharmaceutical agent | House | 6.49 | mg/g | .0649 |
| of formula 1, micronized* | | | | |
| Dibasic Sodium | USP | 4.45 | mg/g | 0.445 |
| Phosphate, Anhydrous | | | | |
| Monobasic Sodium | USP | 1.01 | mg/g | 0.101 |
| Phosphate, Monohydrate | | | | |
| Sucrose | NF | 596 | mg/g | 59.6 |
| Water for Injection | USP | 392 | mg/g | 39.2 |
| Total Target Weight | | 1.00 | g | 100.00% |

*adjusted for purity and moisture content

Manufacturing Process:

The manufacturing process is carried out at room temperature. The phosphates are combined in the water, followed by the sucrose. The resulting solution is sterile filtered and 6.42 g is filled into a 5 mL vial (Type I glass, USP). The vial is closed with a rubber stopper, crimped with an aluminum crimp seal, and terminally sterilized. Prior to use, the solution is rendered isotonic by dilution with Sterile Water for Injection.

### Example 4

| Strength of | Strength of | Final Amount |
|---|---|---|
| Pharmaceutical agent 1 | Ready-to-Used | Delivered in |
| Concentrated Composition | Composition* | Sub-Tenon Injection** |
| 0.83 mg/mL | 0.1 mg/mL | 50 μg |
| 2.5 mg/mL | 0.3 mg/mL | 150 μg |
| 10 mg/mL | 1.2 mg/mL | 60 μg |

*Resulting from 1.0 ML of concentrated composition diluted in 7.34 mL Sterile Water for Injection.
**Injection volume of .05 mL.

The above example illustrates the concentration of the active ingredient Pharmaceutical agent 1 in the concentrated composition and the diluted ready to use composition. The final column illustrates the actual dose. The clinical dosage ready to use concentrations may comprise at least 0.1 mg/ml to at least 1.2 mg/ml. Exemplary dosage concentrations comprise 0.1 mg/ml, 0.3 mg/ml, and 1.2 mg/ml.

It is to be understood that the foregoing description is exemplary and explanatory in nature, and is intended to illustrate the invention and its preferred embodiments. Through routine experimentation, the artisan will recognize apparent modifications and variations that may be made without departing from the spirit of the invention. Thus, the invention is intended to be defined not by the above description, but by the following claims and their equivalents.

We claim:

1. A composition comprising:
(a) an aqueous phase comprising an aqueous solution of a density modifying agent; and
(b) a pharmaceutical agent at least partially in composition in the aqueous phase, wherein the density modifying agent is present in the composition in an amount such that the density of the aqueous phase is substantially the same as the density of the pharmaceutical agent.

2. The composition of claim 1, wherein the amount of the density modifying agent is from 40 to 80 percent by weight, based on the total weight of the aqueous phase.

3. The composition of claim 1, wherein the density modifying agent is sucrose.

4. The composition of claim 1, wherein the density of the aqueous phase is within 10% of the density of the pharmaceutical agent.

5. The composition of claim 1, wherein the pharmaceutical agent is a compound of formula 1 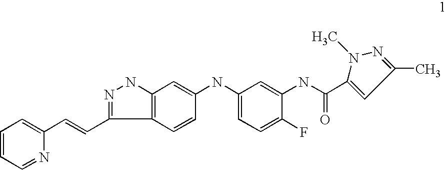
or a pharmaceutically acceptable salt, solvate or hydrate thereof.

6. A method of preparing a pharmaceutical composition, the method comprising forming a composition of a pharmaceutical agent in an aqueous solution, wherein the aqueous solution comprises an amount of a density modifying agent such that the density of the aqueous solution is essentially the same as the density of the pharmaceutical agent.

7. The method of claim 6, wherein the pharmaceutical agent is a compound of formula 1 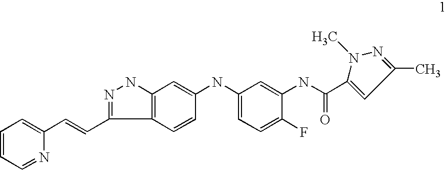
or a pharmaceutically acceptable salt, solvate or hydrate thereof.

8. A method for the periocular delivery of a pharmaceutical agent comprising sub-Tenon administration of the composition of claim 7 to a patient.

9. A composition suitable for administering to a patient comprising:
(a) an aqueous phase comprising an aqueous solution of a density modifying agent; and
(b) a pharmaceutical agent of formula 1 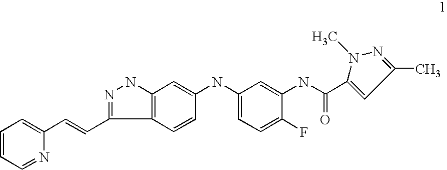
or a pharmaceutically acceptable salt, solvate or hydrate thereof, at least partially in composition in the aqueous phase, wherein the density modifying agent is present in the composition in an amount of from 5 to 15 percent by weight, based on the total weight of the aqueous phase.

10. The composition of claim 9, wherein the composition is suitable for sub-Tenon administration.

11. A method of treating an ophthalmic disease or cancer, comprising administering to a mammal in need thereof a therapeutically effective amount of the composition of claim 9 in combination with radiation.

12. A method of treating an ophthalmic disease or cancer, comprising administering to a mammal in need thereof a therapeutically effective amount of the composition of claim 9 in combination with a chemotherapeutic agent.

13. A method of treating an ophthalmic disease, comprising administering to a mammal in need thereof a therapeutically effective amount of the composition of claim 9 in combination with photodynamic therapy.
